(19) United States
(12) Patent Application Publication
(10) Pub. No.: US 2006/0024701 A1
(43) Pub. Date: Feb. 2, 2006

(54) Methods and reagents for the isolation of nucleic acids

(75) Inventor: Kevin J. McKernan, Marblehead, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 11/126,775

(22) Filed: May 11, 2005

Related U.S. Application Data
(63) Continuation of application No. 10/042,923, filed on Jan. 9, 2002, now abandoned.
(60) Provisional application No. 60/260,774, filed on Jan. 9, 2001.

(51) Int. Cl. C12Q 1/68; C12N 1/08

(52) U.S. Cl. 435/6; 435/270

(57) ABSTRACT

The invention includes reagents and methods for the isolation of nucleic acids. The reagents described herein contain a nucleic acid precipitating agent and a solid phase carrier. The reagents can optionally be formulated to cause the lysis of a cell. These reagents can be used to isolate a target nucleic acid molecule from a cell or a solution containing a mixture of different size nucleic acid molecules. The disclosed reagents and methods provides a simple, robust and readily automatable means of nucleic acid isolation and purification which produces high quality nucleic acid molecules suitable for: capillary electrophoresis, nucleotide sequencing, reverse transcription cloning the transfection, transduction or microinjection of mammalian cells, gene therapy protocols, the in vitro synthesis of RNA probes, cDNA library construction and PCR amplification.

### RELATED APPLICATIONS

This application is a continuation of U.S. application Ser. No. 10/042,923, filed Jan. 9, 2002; which claims the benefit of U.S. Provisional Application No. 60/260,774, filed Jan. 9, 2001. The entire teachings of the above application(s) are incorporated herein by reference.

### STATEMENT AS TO FEDERALLY SPONSORED RESEARCH

This invention was made with Government support under grant number 11-1186-0301 awarded by the National Institutes of Health. The Government may have certain rights in the invention.

### BACKGROUND OF THE INVENTION

Many molecular biology applications, such as capillary electrophoresis, nucleotide sequencing, reverse transcription cloning and gene therapy protocols, which contemplate the transfection, transduction or microinjection of mammalian cells, require the isolation of high quality nucleic acid preparations. Quality is a particularly important factor for capillary electrophoresis for all sequencing methods and for gene therapy protocols. Quantity is an equally important consideration for some applications, for example, large scale genomic mapping and sequencing projects, which require the generation of hundreds of thousands of high quality DNA templates.

Extension product quality is crucial to the success of automated dye labeled dideoxynucleotide sequencing methods, such as those described in Maniatis, T., et al., Molecular Cloning. A Laboratory Manual, 2nd Edition, Cold Spring Harbor Laboratories, Cold Spring Harbor, N.Y., Sanger, E., et al., Proc. Natl. Acad. Sci. 74:5463-5467 (1977), and Mierendorf, R. and Pfeffer, D. Methods Enzymol. 152:5556-562 (1987), and is a particularly critical consideration for capillary electrophoresis protocols. The isolation of high quality nucleic acid preparations from starting mixtures of diverse composition and complexity is a fundamental technique in molecular biology.

The advent of demanding molecular biology applications has increased the need for high-throughput, and preferably readily automatable, purification protocols capable of producing high quality nucleic acid preparations. Although recent technological advancements and the advent of robotics have facilitated the automation of sequencing reactions and gel reading steps, throughput is still limited by the availability of readily automatable methods of nucleic acid purification.

### SUMMARY OF THE INVENTION

The invention is based in part on the discovery that nucleic acids can be purified by using a single reagent to perform steps during the course of the purification process that were formerly performed by using two or more reagents. The reagents and methods described herein thus simplify the process of nucleic acid purification by reducing the number of steps and reagents, thereby easing the automation of the process.

In one aspect the invention includes a method of isolating a first species of nucleic acid molecule from a cell by performing the following steps: (a) providing a cell; (b) preparing a first combination by simultaneously adding to the cell one or more reagent components collectively referred to as a first reagent, wherein the first reagent is formulated to cause lysis of the cell, wherein the first reagent comprises a nucleic acid precipitating reagent and a solid phase carrier having a surface that binds, e.g. reversibly, nucleic acid molecules, wherein the nucleic acid precipitating reagent is present in sufficient concentration to precipitate the first species of nucleic acid molecule; (c) maintaining the first combination under conditions that permit the adsorption of the precipitated nucleic acid molecule to the solid phase carrier, thereby producing a solid phase carrier having bound thereto the first species of nucleic acid molecule; and (d) removing the carrier having bound thereto the first species of nucleic acid molecule from the first combination, thereby isolating the first species of nucleic acid molecule from the mixture and producing a second mixture.

In a preferred embodiment, the first reagent is added to the cell by a multisample transfer device. In another preferred embodiment, the first reagent is added simultaneously to a plurality of samples, e.g., at least 6, 12, 24, 96, 384, or 1536 samples, each sample containing one or more cells. In another preferred embodiment, the first reagent is sequentially delivered to a plurality of samples, e.g., at least 6, 12, 24, 96, 384, or 1536 samples, each sample containing one or more cells.

In a preferred embodiment, the isolated nucleic acid of one or a plurality of samples is subjected to further analysis, e.g., sequence analysis.

A “nucleic acid precipitating reagent” or “nucleic acid precipitating agent” is a composition that causes a nucleic acid molecule to go out of solution. Suitable precipitating agents include alcohols, e.g., short chain alcohols, e.g., ethanol or isopropanol, and a poly-OH compound, e.g., a polyalkylene glycol. Examples of useful polyalkylene glycols include polyethylene glycol (PEG) and polypropylene glycol. In a preferred embodiment, PEG is used, e.g., PEG having an average molecular weight between about 6,000 and about 10,000.

The first reagent can further contain a salt selected from the group consisting of sodium chloride, magnesium chloride, calcium chloride, potassium chloride, lithium chloride, barium chloride and cesium chloride.

A “solid phase carrier” is an entity that is essentially insoluble under any conditions upon which a nucleic acid can be precipitated. The surface can bind, preferably reversibly, a nucleic acid. Suitable solid phase carriers have sufficient surface area to permit efficient binding of nucleic acids. Suitable solid phase carriers include, but are not limited to, microparticles, particles, fibers, beads and or supports on which a precipitated nucleic acid can bind and which can embody a variety of shapes. The shape maximizes the surface area of the solid phase, and embodies a carrier which is amenable to microscale manipulations. In a preferred embodiment, the solid phase carrier is paramagnetic, e.g., a paramagnetic microparticle. In a preferred embodiment, the solid phase carrier includes a functional group coated surface. For example, the solid phase carrier can be an amine-coated paramagnetic microparticle, a carboxyl-coated paramagnetic microparticle, or an encapsulated carboxyl group-coated paramagnetic microparticle.

In carrying out the methods described herein, the pH of the first reagent can be formulated so as to adjust the electronegativity of the solid phase carrier, e.g., the functional group coating the surface of the solid phase carrier, and therefore alter the binding affinity of the solid phase carrier for the first species of nucleic acid molecule. For example, for carboxyl-coated microparticles wherein the carboxy is made with acetic acid, the pKa of the carboxy group is about 4.7. At this pH, the negative charge of the microparticle is neutralized. Below this pH, the microparticle becomes positively charged and less electronegative, thereby making elution from the microparticle more difficult. At higher pHs the carboxy group become more electronegative, thereby facilitating elution of the nucleic acid molecule.

The pH of the first reagent can, for example, be formulated in the range of pH 2.0-11.0, depending upon the desired electronegativity of the solid phase carrier, e.g., the functional group coating the solid phase carrier, and the resulting binding affinity for a nucleic acid molecule. In some embodiments, the pH of the first reagent can be greater than 2, 3, 4, 5, 6, 7, 8, 9, or 10. In other embodiments, the pH can be less than 11, 10, 9, 8, 7, 6, 5, 4, or 3. The pH of the first reagent can be formulated according to the desired ease or difficulty of eluting a nucleic acid molecule from a solid phase carrier.

In one example, the solid phase carrier of the first reagent is removed by applying a magnetic field, applying vacuum filtration, or by centrifugation.

In an additional embodiment, the invention also includes further isolating a second species of nucleic acid molecules from the second mixture describe above. This method contains the additional steps of: (e) preparing a second combination by simultaneously adding to the second mixture one or more reagent components collectively referred to as a second reagent, wherein the second reagent contains a nucleic acid precipitating reagent and a solid phase carrier having a surface that binds, e.g., reversibly, nucleic acid molecules, wherein the precipitating reagent is present in sufficient concentrations to precipitate the second species of nucleic acid molecule; (f) maintaining the second combination under conditions appropriate for the adsorption of the second target species of nucleic acid molecule to the surface of the solid phase carrier, thereby producing a solid phase carrier having the second species of nucleic acid molecule bound thereto; and (g) removing the solid phase carrier having the second species of nucleic acid molecule adsorbed thereto from the second combination. This method can optionally include the additional step of (h) eluting the second species of nucleic acid molecules from the solid phase carrier, thereby selectively isolating an second species of nucleic acid molecules.

In a preferred embodiment, the second reagent is added to the second mixture by a multisample transfer device. In another preferred embodiment, the second reagent is added simultaneously to a plurality of samples, e.g., at least 6, 12, 24, 96, 384, or 1536 samples. In another preferred embodiment, the second reagent is sequentially delivered to a plurality of samples, e.g., at least 6, 12, 24, 96, 384, or 1536 samples.

According to these methods, the second species of nucleic acid can be of a smaller molecular size than the first species removed in step (d). The nucleic acid precipitating reagent and solid phase carrier can have the properties described herein. In addition, the pH of the second reagent can be formulated so as to adjust the electronegativity of the solid phase carrier as described herein with respect to the first reagent, so as to modulate the ease or difficulty of eluting the second species of nucleic acid from the solid phase carrier. The solid phase carrier of the second reagent can removed by applying a magnetic field, applying vacuum filtration, or by centrifugation.

In one embodiment, the first reagent comprises a nucleic acid precipitating reagent, e.g., polyethylene glycol, in a concentration that results in the binding of the first species of nucleic acid molecule to the solid phase carrier in step (c), but does not result in the binding of the second species of nucleic acid molecule to the solid phase carrier in step (c).

In another embodiment, the polyethylene glycol has an average molecular weight of about 8,000, and the polyethylene glycol concentration of the first combination is between about 1% and 4% and the polyethylene glycol concentration of the second combination is at least 7%.

In another aspect, the invention includes a method of isolating a nucleic acid molecule from a cell by adding to the cell one or more reagent components collectively referred to as a first reagent, wherein the first reagent causes the lysis of the cell and contains a nucleic acid precipitating reagent and a solid phase carrier having a surface that reversibly binds a nucleic acid molecule of the cell.

In a preferred embodiment, the first reagent is added to the cell by a multisample transfer device. In another preferred embodiment, the first reagent is added simultaneously to a plurality of samples, e.g., at least 6, 12, 24, 96, 384, or 1536 samples, each sample containing one or more cells. In another preferred embodiment, the first reagent is sequentially delivered to a plurality of samples, e.g., at least 6, 12, 24, 96, 384, or 1536 samples, each sample containing one or more cells.

In a preferred embodiment, the isolated nucleic acid of one or a plurality of samples is subjected to further analysis, e.g., sequence analysis.

In one embodiment, the method further includes removing the solid phase carrier with a first species of nucleic acid attached thereto, to generate a first mixture. The method also includes adding to the first mixture one or more reagent components collectively referred to as a second reagent, wherein the second reagent includes a nucleic acid precipitating agent and a solid phase carrier having a surface that reversibly binds a nucleic acid molecule of the cell.

In a preferred embodiment, the isolated nucleic acid of one or a plurality of samples is subjected to further analysis, e.g., sequence analysis.

In another aspect, the invention includes methods of analyzing a plurality of nucleic acid samples. The methods include providing a plurality of nucleic acid samples isolated by a method described herein and analyzing the samples, e.g., performing sequence analysis on the samples.

In another aspect, the invention includes compositions used in the methods described herein. In one aspect, the invention includes a composition for isolating nucleic acids, wherein the composition contains a nucleic acid precipitating reagent and a solid phase carrier having a surface that binds nucleic acid molecules, wherein the composition is formulated to cause the lysis of a cell, wherein the composition lacks one or more of nucleic acids, cells, or cellular lysate.

In one example, the composition contains polyethylene glycol and salt. For example the concentration of PEG can be formulated to be between about 1-4% when the composition is added to a cell. In addition, the concentration of salt can be formulated to be at least 0.5M when the composition is added to a cell.

In a preferred embodiment, the PEG and salt are present in sufficient concentration to selectively precipitate nucleic acid molecules greater than a predetermined size, e.g., 5, 6, 7, 8, 9, 10, 15, 20, 50, 100, 150, 200, 250, 300, 350, 400, 450, or 500 kilobases, when the composition is added to a cell.

In one embodiment, the PEG and salt are present in sufficient concentration to selectively precipitate nucleic acid molecules greater 10 kb when the composition is added to a cell.

As described herein with respect to reagents used in methods of the invention, the pH of the composition can be formulated so as to adjust the electronegativity of the surface of the solid phase carrier, so as to modulate the binding affinity of the surface of the solid phase carrier to nucleic acid molecules.

In another aspect, the invention includes a composition for isolating nucleic acids, wherein the composition contains a nucleic acid precipitating reagent and a solid phase carrier having a surface that binds, preferably reversibly, nucleic acid molecules, wherein the composition lacks one or more of nucleic acids, cells, or cellular lysate.

In one example, the composition contains polyethylene glycol and salt. For example the concentration of PEG is formulated to be at least 7% when the composition is added to a cell. In addition, the concentration of salt can be formulated to be between less than 055M when the composition is added to a cell.

In a preferred embodiment, the PEG and salt are present in sufficient concentration to selectively precipitate nucleic acid molecules greater than a predetermined size, e.g., 1, 10, 50, 100, or 500 base pairs, or 1, 1.5, 2, 2.3, 2.4 2.5, 3, 4, 5, 6, 7, 8, 9, 10, 15, 20, 50, 100, 150, 200, 250, 300, 350, 400, 450, or 500 kilobases, when the composition is added to a cell.

In one embodiment, the PEG and salt are present in sufficient concentration to selectively precipitate nucleic acid molecules greater 2.4 kb when the composition is added to a cell.

In another aspect, the invention includes a kit for isolating nucleic acids that contains: a first composition, wherein the first composition includes a nucleic acid precipitating reagent and a solid phase carrier having a surface that binds nucleic acid molecules, wherein the first composition is formulated to cause the lysis of a cell, wherein the first composition lacks one or more of nucleic acids, cells, or cellular lysate; and a second composition, wherein the second composition includes a nucleic acid precipitating reagent and a solid phase carrier having a surface that reversibly binds nucleic acid molecules, wherein the second composition lacks one or more of nucleic acids, cells, or cellular lysate.

In one embodiment, the kit additionally includes a third composition and a fourth composition, wherein the third composition dissolves impurities but not nucleic acids bound to a solid phase carrier, and wherein the fourth composition is a low ionic strength buffer.

A kit described herein can optionally include a magnetic sample container, e.g., plate, holder appropriate for applying a magnetic field, preferably of at least about 1000 Gauss, to the sample container, e.g., to the wells of a microtiter plate. The magnet can include at least one N35 magnet.

The present invention is useful to isolate, from a mixture from which at least one species of nucleic acid molecule has been selectively removed, one or more additional (e.g., a second, third, fourth etc.) species of nucleic acid molecules which are of a smaller molecular size than the one or more target nucleic acid species which have already been removed from an initial (or starting) mixture by the method described herein. The additional species of nucleic acid molecule targeted for isolation in this additional embodiment remained soluble in the presence of the PEG and salt concentrations used to isolate the larger nucleic acid molecule and, therefore, will still be present in the mixture from which the first target nucleic acid molecule has been removed.

In an alternative embodiment of the instant invention, two or more species of nucleic acid molecule present in the same mixture, which differ in molecular size from each other by at least a factor of two, are separated from each other. The method described herein is used to isolate a particular species (e.g., a target species) of nucleic acid molecules of virtually any size, present in a wide variety of sources, from other nucleic acid molecules which are also present in the mixture. For example, the method disclosed herein can be used to isolate recombinant nucleic acid species, produced in host cells, including selective RNA precipitations based on molecular size, or replicative form of DNA produced by a virus during lytic replication from endogenous host cell nucleic acid species. The method can also be used to isolate a particular species of nucleic acid from a solution resulting from a restriction enzyme digestion or an agarose solution containing nucleic acid. Alternatively, the method disclosed herein provides a size selection purification scheme suitable for use after a DNA shearing process (e.g., hydroshearing or sonication), thereby providing an alternative to the more traditional method of gel electrophoresis and band excision which are conventionally used to isolate a species of nucleic acid molecule targeted for purification. The disclosed method also finds utility as a method of separating multiplex PCR products, or as a sequencing reaction detemplating protocol. For example, using the method disclosed herein solid phase magnetically responsive paramagnetic microparticles can be used to selectively remove sequencing products and DNA templates from sequencing samples.

The present invention is also useful to isolate, from a mixture from which at least one species of nucleic acid molecule has been selectively removed, one or more additional (e.g., a second, third, fourth etc.) species of nucleic acid molecules which are of a smaller molecular size than the one or more target nucleic acid species which have already been removed from an initial (or starting) mixture by the method described herein. The additional species of nucleic acid molecule targeted for isolation in this additional embodiment remained soluble in the presence of the PEG and salt concentrations used to isolate the larger nucleic acid molecule and, therefore, will still be present in the mixture from which the first target nucleic acid molecule has been removed.

The present invention further relates to a method of isolating an exogenous DNA template (e.g., a plasmid DNA template) suitable for use in either manual or a high-throughput automated sequencing methods. In general terms this method comprises: treating host cells which contain exogenous DNA (e.g., plasmid DNA) with a first reagent as describe herein, wherein the nucleic acid precipitating agent is in sufficient concentration to selectively precipitate and adsorb host cell DNA (e.g., genomic DNA), but not exogenous DNA, to the surfaces of the solid phase carrier; removing the microparticles having host cell DNA bound thereto from the suspension, preferably by magnetic means, thereby producing a plasmid DNA-enriched supernatant; combining a second reagent as described herein with the resulting plasmid DNA enriched supernatant; and adjusting the precipitating reagent and/or salt concentration of this supernatant to suitable levels to result in the selective precipitation and adsorption of exogenous (e.g., plasmid) DNA to the microparticles suspended therein. As a result, exogenous (e.g., plasmid) DNA is bound to the microparticles, thereby producing microparticle-bound exogenous DNA.

The purity of the microparticle-bound exogenous DNA can be improved by washing the particle-bound nucleic acid molecules to remove other host cell biomolecules by contacting the microparticles with a high ionic strength wash buffer which dissolves, for example impurities (e.g., proteins, reagents or chemicals) adsorbed to the paramagnetic microparticles, but does not solubilize the adsorbed DNA. As a result, the exogenous DNA targeted for isolation remains adsorbed to the solid phase carrier surface. The washed, particle-bound exogenous DNA template can subsequently be removed from the solid phase carrier by contacting the washed microparticles with an elution buffer which solubilizes the adsorbed DNA, thereby preparing plasmid DNA suitable for use as a DNA nucleotide sequencing template.

In one embodiment, the invention is a readily automatable method of isolating a plasmid DNA template for nucleotide sequencing. The use of the reagents described herein affords an alternative, and readily automatable, means of molecular separation useful in the design of a solid phase technique for the selective isolation of nucleic acid molecules targeted for isolation.

The present invention also relates to a kit comprising magnetically responsive microparticles preferably having a functional group-coated surface that binds, preferably reversibly, nucleic acid molecules, at least one binding buffer, a suitable nucleic acid precipitating reagent and salt at concentrations suitable for reversibly binding nucleic acids onto the surface of the microparticle. The kit may additionally comprise preformulated solutions of a host cell lysis buffer, or reagents for the preparation of such buffer, a wash buffer and an elution buffer. The exact compositions of the buffers may vary with the nature of the starting material and the purpose (e.g., the molecular biology application) for which the nucleic acid preparation is being isolated. The kit may further include a magnetic microtiter plate holder specifically designed to optimize the field strength applied to remove the paramagnetic microparticles from the resulting combinations and solutions.

An advantage of the invention is that it allows for a simplified procedure for purifying nucleic acids. By providing a single reagent, described herein as a first reagent, that causes lysis of cells and contains a nucleic acid precipitating agent and a solid phase carrier, one or more steps can be removed from the standard purification process. For example, traditional alkaline lysis requires the following steps: lysis of cells with alkaline detergent; shaking and or agitation; addition of neutralization buffer and filter; addition of a solid phase carrier; and addition of binding buffer. The methods described herein allow for the addition of a single reagent to a cell, followed by an incubation and a separation of a solid phase carrier. No pH adjustments are required by the methods of the invention. The reduced number of steps provided by the reagents and methods described herein simplifies the automation of the nucleic acid purification process.

In addition, the elimination of the alkaline lysis procedure allows for the purification of a high percentage, e.g., at least 90%, of supercoiled plasmid DNA.

Another advantage of the invention is that it allows for long term sterile storage of a solid phase carrier, e.g., magnetic beads. Magnetic beads with negatively charged functional groups will bind DNA and other biomolecules rapidly. Traditional storage buffers permit cell growth. Cell growth produces biomolecule accumulation in the storage solution and hence expiration of bead binding area. Reagents described herein, where the beads and binding buffer are dissolved in a compatible lysis buffer (lysis buffers by definition are free of cell growth) allow long term storage and are very attractive kit features.

A further advantage of the invention is that the reagents and methods described herein allow for simple and accurate nanodispensing of fluids. As a result this procedure can be reliably performed in formats which handle a relatively large number of samples, e.g., at least 6, 12, 24, 48, 96, 384, or 1536.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts the sequencing results of a 5 kb plasmid DNA insert.

### DETAILED DESCRIPTION OF THE INVENTION

The present invention provides methods and reagents for isolating nucleic acids. The reagents described herein can be used to isolate nucleic acid from a cell by the simultaneous addition to the cell of a first reagent comprising a nucleic acid precipitating agent and a solid phase carrier, wherein the first reagent is formulated to cause the lysis of the cell. The invention also includes reagents and methods for isolating nucleic acids by using a second reagent that comprises a nucleic acid precipitating agent and a solid phase carrier.

The isolation of high quality nucleic acid preparations from starting solutions of diverse composition and complexity is a fundamental technique in molecular biology. Thus, as a result of the reagents and methods described herein, rapid and readily automatable methods of isolating and purifying nucleic acid molecules are now available. Nucleic acids isolated by the disclosed methods can be used for molecular biology applications requiring high quality nucleic acids, such as the preparation of DNA sequencing templates, microinjection, transfection or transformation of mammalian cells, in vitro synthesis of RNA probes, reverse transcription cloning, cDNA library construction, PCR amplification, and gene therapy research, as well as for other applications with less stringent quality requirements including, but not limited to, transformation, restriction endonuclease or microarray analysis, selective RNA precipitations, in vitro transposition, separation of multiplex PCR amplification products, preparation of DNA probes and primers and detemplating protocols.

The reagents and methods described herein can be used together with a variety of nucleic acid purification techniques, including those described in U.S. Pat. Nos. 5,705,628 and 5,898,071 as well as WO 99/58664, the contents of which are herein incorporated by reference.

Methods of Isolating Nucleic Acids

One embodiment of the instant invention comprises methods of isolating a first species of nucleic acid molecule from a cell by adding to the cell a first reagent that contains a nucleic acid precipitating agent and a solid phase carrier, wherein the first reagent is formulated to cause the lysis of the cell. The components of this first reagent can be contained in one or more reagents. When contained in more than one reagent, the components are added to the cell simultaneously. Preferably, the components are contained in one reagent. The nucleic acid precipitating agent is in sufficient concentration to precipitate the first species of nucleic acid molecule. The solid phase carrier in this first reagent contains a surface that binds the first species of nucleic acid molecule. The combination (first combination) generated by the addition of the first reagent to the cell is maintained under conditions appropriate for adsorption of the precipitated nucleic acid molecules to the surface of the solid phase carrier, thereby producing a solid phase carrier having the first species of nucleic acid molecule bound thereto.

Suitable precipitating agents include ethanol, isopropanol and polyalkylene glycols. In a preferred embodiment, PEG is used. Suitable PEG can be obtained from Sigma (Sigma Chemical Co., St. Louis Mo., Molecular weight 8000, Dnase and Rnase fee, Catalog number 25322-68-3). The molecular weight of the polyethylene glycol (PEG) can range from about 6,000 to about 10,000, from about 6,000 to about 8,000, from about 7,000 to about 9,000, from about 8,000 to about 10,000. In particular embodiment PEG with a molecular weight of about 8,000 is used. In general, the presence of PEG provides a hydrophobic solution which forces hydrophilic nucleic acid molecules out of solution. The advantages of using PEG which is a nondenaturing water soluble polymer, rather than an organic precipitating reagent (e.g., ethanol, isopropanol or phenol), are attributed to its benign chemical properties.

According to the current invention, nucleic acid precipitates, e.g., PEG-induced nucleic acid precipitates, are adsorbed to the surfaces of a solid phase carrier, e.g., a magnetically responsive microparticle, which can be physically manipulated to facilitate the isolation of nucleic acid molecules from complex solutions comprising mixtures of nucleic acids, in the presence or absence of other host cell biomolecules. Although numerous biological macrostructures (bacteriophage, ribosomes, plant and animal viruses, proteins and nucleic acids) are precipitable with PEG, the threshold concentration required varies for each macrostructure (Lis, Methods in Enzymology, 1980). This observation makes it possible to use the instant method to isolate nucleic acid molecules, not only from other nucleic acid molecules having a different molecular size, but also from other host cell biomolecules and biological macro structures, each of which will have a distinct PEG threshold concentration at which it will precipitate.

The first reagent can also contain salts to facilitate the adsorption of the nucleic acid to the solid phase carrier. Suitable salts which are useful for facilitating the adsorption of nucleic acid molecules targeted for isolation to a solid phase carrier, e.g., a magnetically responsive microparticle, include sodium chloride (NaCl), lithium chloride (LiCl), barium chloride (BaCl₂), potassium (KCl), calcium chloride (CaCl₂), magnesium chloride (MgCl₂) and cesium chloride (CsCl). In a preferred embodiment, sodium chloride is used. In general, the presence of salt functions to minimize the negative charge repulsion of the nucleic acid molecules. The wide range of salts suitable for use in the method indicates that many other salts can also be used and suitable levels can be empirically determined by one of ordinary skill in the art.

As used herein, “facilitated adsorption” refers to a process whereby a nucleic acid precipitating reagent, (e.g., a poly-alkyelene glycol) is used to promote the precipitation and subsequent adsorption of a species of DNA molecules, which were initially in mixture, onto the surface of a solid phase carrier. The resulting reversible interaction is distinct from, for example, an interaction between two binding partners (e.g., streptavidin/biotin, antibody/antigen, or a sequence-specific interaction), which are conventionally utilized for the purpose of isolating particular biomolecules based on their composition or sequence.

As used herein, “paramagnetic solid phase carrier” refers to an entity which responds to an external magnetic field (e.g., a plastic tube or a microtiter plate holder) with an embedded rare earth (e.g., neodymium) magnet but which demagnetize when the field is removed.

As used herein, “paramagnetic microparticles” refer to microparticles which respond to an external magnetic field (e.g., a plastic tube or a microtiter plate holder) with an embedded rare earth (e.g., neodymium) magnet but which demagnetize when the field is removed. Thus, the paramagnetic microparticles are efficiently separated from a solution using a magnet, but can be easily resuspended without magnetically induced aggregation occurring. Preferred paramagnetic microparticles comprise a magnetite rich core encapsulated by a pure polymer shell. Suitable paramagnetic microparticles comprise about 20-35% magnetite/encapsulation ratio. For example, magnetic particles comprising a magnetite/encapsidation ration of about 23%, 25%, 28% 30% 32% or 34% are suitable for use in the present invention. Magnetic particles comprising less than about a 20% ratio are only weakly attracted to the magnets used to accomplish magnetic separations. Depending on the nature of the host cell, the viscosity of the cell growth and the nature of the vector (e.g. high or low copy) paramagnetic microparticles comprising a higher percentage of magnite should be considered. The use of encapsulated paramagnetic microparticles, having no exposed iron, or Fe₃O₄ on their surfaces, eliminates the possibility of iron interfering with polymerase function in certain downstream manipulations of the isolated DNA. However the larger the magnetite core the higher the chance of encapsulation leakage (e.g., release of iron oxides). Suitable paramagnetic microparticles for use in the instant invention can be obtained for example from Bangs Laboratories Inc., Fishers, Ind. (e.g., estapor® carboxylate modified encapsulated magnetic microspheres).

Suitable paramagnetic microparticles should be of a size that their separation from solution, for example by magnetic means or by filtration, is not difficult. In addition, preferred paramagnetic microparticles should not be so large that their surface area is minimized or that they are not suitable for microscale manipulation. Suitable sizes range from about 0.1 μm mean diameter to about 100 μm mean diameter. A preferred size is about 1.0 μm mean diameter.

As described above, the first reagent is formulated to cause the lysis of a cell. A variety of lysis components can be used to cause the disruption of a membrane (such as alkali, alkali and anionic detergent treatment, or isotonic shock). In one embodiment, the lysis component of the first reagent is an alkali and anionic detergent (e.g., sodium dodecyl sulphate (SDS)) solution (e.g., final concentration of 0.2 N NaOH, 1% SDS when added to a cell). Optionally, lysozyme could be included in the lysis component of the first reagent. The presence of an anionic detergent in the lysis component functions to produce an anti-protein environment by neutralizing the effective charge of the proteins, thereby minimizing their attraction to the surfaces of the solid phase carrier, e.g., a functional group-coated paramagnetic microparticle. Optionally, RNase can be added to the lysis component to degrade host cell RNA, thereby allowing DNA to bind to the solid phase carrier free, or essentially free, from RNA. The necessity of including a RNase step will largely be determined by the size of the nucleic acid species that is targeted for isolation in the particular nucleic acid precipitation that is being performed. For example, if the conditions selected for isolation are appropriate for isolating nucleic acids comprising at least 4,000 base pairs, then it is unlikely that RNA species will be an appreciable contaminant.

As used herein, the term “functional group-coated surface” refers to a surface which is coated with moieties which reversibly bind nucleic acid (e.g., DNA, RNA or polyamide nucleic acids (PNA)). One example is a surface which is coated with moieties which each have a free functional group which is bound to the amino group of the amino silane or the microparticle; as a result, the surfaces of the microparticles are coated with the functional group containing moieties. The functional group acts as a bioaffinity adsorbent for polyalkylene glycol precipitated DNA. In one embodiment, the functional group is a carboxylic acid. A suitable moiety with a free carboxylic acid functional group is a succinic acid moiety in which one of the carboxylic acid groups is bonded to the amine of amino silanes through an amide bond and the second carboxylic acid is unbonded, resulting in a free carboxylic acid group attached or tethered to the surface of the paramagnetic microparticle. Suitable solid phase carriers having a functional group coated surface that reversibly binds nucleic acid molecules are for example, magnetically responsive solid phase carriers having a functional group-coated surface, such as, but not limited to, amino-coated, carboxyl-coated and encapsulated carboxyl group-coated paramagnetic microparticles.

As used herein the terms “nucleic acid” and “nucleic acid molecule” are used synonymously with the term polynucleotides and they are meant to encompass DNA (single-stranded, double-stranded, covalently closed, and relaxed circular forms), RNA (single-stranded and double-stranded), RNA/DNA hybrids and polyamide nucleic acids (PNAs).

The term “species” as it used herein to refer to nucleic acid molecules means a particular subclass, family or type of nucleic acid molecule defined on the basis of a characteristic size. Thus, the members of a “species of nucleic acid molecules” are all of approximately equivalent molecular size within a small range of molecule sizes.

As used herein the term “isolated” is intended to mean that the material in question exists in a physical milieu distinct from that in which it occurs in nature and/or has been completely or partially separated or purified from other nucleic acid molecules.

According to the methods of the invention, the isolation of the first species of nucleic acid molecules is accomplished by removing the nucleic acid-coated solid phase carrier from the first combination. The solid phase carrier (e.g., a paramagnetic microparticle) can be recovered from the first combination, for example, by vacuum filtration, centrifugation, or by applying a magnetic field to draw down the solid phase carrier, e.g., a paramagnetic microparticle. Paramagnetic microparticles are preferably separated from solutions using magnetic means, such as applying a magnet field of at least 1000 Gauss. However, other methods known to those skilled in the art can be used to remove the magnetic microparticles from the supernatant (e.g., vacuum filtration or centrifugation). The remaining solution can then be removed, leaving solid phase carriers having a particular nucleic acid species adsorbed to their surface.

Once separated from the mixture, the isolated nucleic acid species adsorbed to the solid phase carrier can be recovered by contacting the solid phase carrier with a suitable elution buffer. As a result, a solution comprising the target nucleic acid molecules and solid phase carrier is produced. Using appropriate means, for example, magnetic means, the solid phase carriers are subsequently removed from the solution whereby the target species of nucleic acid molecule is isolated from the mixture and a second mixture is produced.

A suitable elution buffer can be water or any aqueous solution in which the salt concentration and nucleic acid precipitating agent, e.g., PEG, concentration are below the concentrations required for binding of DNA to the solid phase carrier, as discussed above. For example, useful buffers include, but are not limited to, TRIS-HCl, Tris acetate, sucrose (20%) and formamide (100%) solutions. Elution of the DNA from the solid phase carrier can occur quickly (e.g., in thirty seconds or less) when a suitable low ionic strength elution buffer is used. Once the bound DNA has been eluted, the solid phase carrier is separated from the elution buffer.

Optionally, impurities (e.g., host cell components, proteins, metabolites or cellular debris) can be removed by washing the solid phase carrier with target nucleic acid bound thereto (e.g., by contacting the solid phase carrier with a suitable wash buffer solution) before separating the solid phase carrier-bound nucleic acid from the solid phase carrier. The composition of the wash buffer is chosen to ensure that impurities either bound directly to the solid phase carrier, or associated with the adsorbed DNA are dissolved. The pH and solute composition and concentration of the wash buffer can be varied according to the types of impurities which are expected to be present. For example, ethanol exemplifies a preferred wash buffer useful to remove excess PEG and salt. The solid phase carrier with bound DNA can also be washed with more than one wash buffer solution. The solid phase carrier can be washed as often as required (e.g., three to five times) to remove the desired impurities. However, the number of washings is preferably limited to in order to minimize loss of yield of the bound DNA. A suitable wash buffer solution has several characteristics. First, the wash buffer solution must have a sufficiently high salt concentration (a sufficiently high ionic strength) that the nucleic acid bound to the solid phase carrier does not elute off of the solid phase carrier, but remains bound. A suitable salt concentration is greater than about 0.1 M and is preferably about 0.5M. Second, the wash buffer solution is chosen so that impurities that are bound to the DNA or solid phase carrier are dissolved. The pH and solute composition and concentration of the buffer solution can be varied according to the types of impurities which are expected to be present. Suitable wash solutions include the following: 0.5×5 SSC; 100 mM ammonium sulfate, 400 mM Tris pH 9, 25 MM MgCl₂ and 1% bovine serum albumin (BSA); and 0.5M NaCl. A preferred wash buffer solution comprises 25 mM Tris acetate (pH 7.8), 100 mM potassium acetate (KOAc), 10 mM magnesium acetate (Mg₂OAC), and 1 mM dithiothreital (DTT).

In an additional embodiment, the invention comprises methods for isolating a second species of nucleic acid molecules from the second mixture (produced by the removal of nucleic acid-coated solid phase carrier from the first combination above). In this embodiment, the second species of nucleic acid molecule can be of a smaller molecular size than the first target species isolated from the first combination. More specifically, this method comprises the steps of producing a second combination by adding to the second mixture a second reagent that contains a nucleic acid precipitating agent and a solid phase carrier. The components of this second reagent can be contained in one or more reagents. When contained in more than one reagent, the components are added to the cell simultaneously. Preferably, the components are contained in one reagent. The nucleic acid precipitating agent is in sufficient concentration to precipitate the second species of nucleic acid molecule (as compared to the first combination above created by the addition of the first reagent, which does not result in the precipitation of the second species of nucleic acid molecule). The solid phase carrier in this second reagent contains a surface that binds the first species of nucleic acid molecule.

The second combination is maintained under conditions appropriate for the absorption of the second target species to the surface of the solid phase carrier, thereby producing a solid phase carrier having the second target species of nucleic acid molecule bound thereto. Isolation is accomplished by removing the solid phase carrier having the second species of nucleic acid molecules absorbed thereto from the second combination and eluting the additional target species into a suitable low ionic strength solution. A wash buffer may optionally be applied prior to eluting the nucleic acid, using the same criteria as described above with respect to the first species of nucleic acid.

In the embodiments described above, a first and or second species of nucleic acid is isolated from a cell. In these previously described methods, suitable starting materials can be host cells containing an exogenous nucleic acid (e.g., recombinant DNA, bacterial DNA or replicative form DNA) which is targeted for isolation from host cell chromosomal DNA and other host cell biomolecules. According to those methods, host cells are lysed using the first reagent, thereby resulting in the attachment of a first species of nucleic acid to a solid phase carrier. A second species of nucleic acid molecule can then optionally be isolated by using the second reagent.

The invention also includes methods and reagents useful for the isolation of nucleic acids from a variety of starting materials in addition to cells. Appropriate starting materials include, but are not limited to, lysates prepared from cells obtained from either mammalian tissue or body fluids, nucleic acid samples eluted from agarose or polyacrylamide gels, solutions containing multiple species of DNA molecules resulting either from a polymerase chain reaction (PCR) amplification or from a DNA size selection procedure and solutions resulting from a post-sequencing reaction. Suitable starting solutions typically are mixtures of biomolecules (e.g. proteins, polysaccharides, lipids, low molecular weight enzyme inhibitors, oligonucleotides, primers, templates) and other substances such as agarose, polyacrylamide, trace metals and organic solvents, from which the target nucleic acid molecule must be isolated.

According to these methods, a combination is produced by adding to the starting material a second reagent that contains a nucleic acid precipitating agent and a solid phase carrier. This second reagent is the same as that described in previous methods. The components of this second reagent can be contained in one or more reagents. When contained in more than one reagent, the components are added to the starting material simultaneously. Preferably, the components are contained in one reagent. The nucleic acid precipitating agent is in sufficient concentration to precipitate the target species of nucleic acid molecule. The solid phase carrier in this second reagent contains a surface that binds the target species of nucleic acid molecule. The method optionally includes washing and or elution steps, as described above.

For example, a mixture of nucleic acids can be separated, according to methods known to one skilled in the art (e.g., gel electrophoresis), such as by agarose gel electrophoresis. A plug of agarose containing nucleic acid on interest can be excised from gel and combined with an appropriate buffer, into which the nucleic acid is released by heating the combination to dissolve the agarose plug. The method of the instant invention can also be used to separate a particular species of DNA present in a post-shearing procedure mixture or to remove a template and primers from a sequencing reaction or to separate PCR primers from the reaction product of a PCR amplification protocol.

Reagents for Isolating Nucleic Acids

The invention provides several reagents for use in isolating nucleic acids. These reagents can be used in methods to isolate nucleic acids either from cells or from other non-cellular starting materials, such as those described herein.

The first reagent used in the methods described above is useful for isolating a nucleic acid from a cell. This first reagent contains a nucleic acid precipitating agent and a solid phase carrier, and is formulated to cause the lysis of a cell. As described above, the components of the first reagent are preferably contained in one reagent. The nucleic acid precipitating agent is in sufficient concentration to precipitate the first species of nucleic acid molecule. The solid phase carrier in this first reagent contains a surface that binds the first species of nucleic acid molecule. The nature and quantity of the components contained in the first reagent are as described in the methods above. The first reagent may formulated in a concentrated form, such that dilution is required to obtain the functions and or concentrations described in the methods herein. This first reagent preferably lacks one or more of nucleic acids, cells, or cellular lysate.

The second reagent used in the methods described above is useful for isolating a nucleic acid from noncellular starting material, e.g., a cell lysate, such as a cell lysate that has been treated with the first reagent. This second reagent contains a nucleic acid precipitating agent and a solid phase carrier. As described above, the components of the second reagent are preferably contained in one reagent. The nucleic acid precipitating agent is in sufficient concentration to precipitate the second species of nucleic acid molecule. The solid phase carrier in this second reagent contains a surface that binds the second species of nucleic acid molecule. The second reagent may formulated in a concentrated form, such that dilution is required to obtain the functions and or concentrations described in the methods herein. This second reagent preferably lacks one or more of nucleic acids, cells, or cellular lysate.

Selective Isolation of Nucleic Acid Molecules

One embodiment of the instant invention is a method of selectively isolating a target species of nucleic acid molecule, on the basis of its molecular size, from a solution comprising a mixture of the target nucleic acid species in the presence or absence of other species of nucleic acid molecules and other biomolecules. As described herein, the method comprises preparing a combination comprising a mixture of nucleic acids in the presence of a reagent described herein, e.g., a first and second reagent, containing a solid phase carrier and a nucleic acid precipitating reagent, e.g., PEG, wherein the nucleic acid precipitating reagent concentration is sufficient to selectively precipitate a particular species of nucleic acid molecule, which has been targeted for isolation.

As used herein the terms “selective” and “selectively” refer to the ability to isolate a particular species of DNA molecule, on the basis of molecular size (e.g., host cell chromosomal DNA or exogenous plasmid DNA), from a combination which includes or is a mixture of species of DNA molecules, such as a host cell lysate and other host cell components. The selective isolation of a particular species is accomplished through the use of an appropriate nucleic acid precipitating reagent (e.g., polyalkylene glycol) to result in the precipitation and facilitated adsorption of a particular DNA species (e.g., characterized on the basis of size) to the surfaces of a solid phase carrier, e.g., a paramagnetic microparticle.

The nucleic acid precipitating reagent, e.g., PEG, should be present at levels which are sufficient to precipitate the targeted nucleic acid species, but insufficient to precipitate relatively smaller sized nucleic acid molecules or other host cell biomolecules. The precipitated nucleic acid species targeted for isolation is removed from the solution by adding a solid phase carrier, such as a magnetically responsive microparticle, which has a surface, e.g., a functional group-coated surface, that binds, e.g., reversibly, nucleic acid molecules to its surfaces. The nucleic acid-coated carrier represents a solid phase product which can subsequently be removed from the starting solution by the application of an external force (e.g., centrifugation, filtration or magnetic field).

The removal of the solid phase carrier from the solution, results in the isolation of a target species of nucleic acid molecule, characterized by a particular molecular size, which is essentially free of other host cell biomolecules and as a consequence produces a solution from which nucleic acids characterized by a particular molecular size have been removed. An second species of nucleic acid molecule (or a third, fourth, fifth, etc.) characterized by having a relatively smaller molecular size can subsequently be isolated from the resulting solution by adding a solid phase carrier, having a functional group-coated surface that reversibly binds nucleic acid molecules, to the solution (from which nucleic acid molecules of relatively higher molecular weight have been removed) in the presence of sufficient polyethylene glycol and salt to precipitate the relatively smaller species of nucleic acid molecule subsequently targeted for isolation. The resulting combination is maintained under conditions which favor the adsorption of the second nucleic acid species, but not other host cell biomolecules present in the solution, to the surfaces of the microparticles, thereby producing a second solid phase product. The removal of the second solid phase product from the solution results in the isolation of an additional species of nucleic acid molecule that is essentially free of other species of nucleic acid molecules, characterized by different molecular sizes, and of other biomolecules present in the starting solution.

As used herein a “host cell” is any cell into which exogenous DNA can be introduced, producing a host cell which contains exogenous DNA, in addition to host cell DNA. As used herein the terms “host cell DNA” and “endogenous DNA” refer to DNA species (e.g., genomic or chromosomal DNA) that are present in a host cell as the cell is obtained. As used herein, the term “exogenous” refers to DNA other than host cell DNA; exogenous DNA can be present into a host cell as a result of being introduced in the host cell or being introduced into an ancestor of the host cell.

Thus, for example, a DNA species which is exogenous to a particular host cell is a DNA species which is non-endogenous (not present in the host cell as it was obtained or an ancestor of the host cell). Appropriate host cell include, but are not limited to, bacterial cells, yeast cells, plant cells and mammalian cells.

The term “lysed host cell suspension”, as used herein, refers to a suspension comprising host cells whose membranes have been disrupted by any means (e.g., chemical, such as alkali or alkali and anionic detergent treatment, isotonic shock, or physical disruption by homogenization), such a suspension is a mixture of host cell biomolecules, cellular components and disrupted membrane debris. In one embodiment, a lysed host cell suspension suitable for use in the instant invention is prepared by contacting host cells with an alkali and anionic detergent (e.g., SDS) solution (e.g., 0.2 N NaOH, 1% SDS). Optionally, lysozyme could be included in the lysis buffer. In one embodiment, the lysed host cell suspension is non-neutralized. Optionally, RNase can be added to the host cell lysate to degrade host cell RNA, thereby allowing DNA to bind to the magnetic microparticles free, or essentially free, from RNA.

In one embodiment, the present invention provides a method of selectively isolating a species of nucleic acid molecule present in a mixture from other nucleic acid molecules, and from other biomolecules, biological macro structures, or reagents possibly present in the starting material. More specifically, this embodiment of the method involves combining a first or second reagent described herein with a mixture which comprises the target species of nucleic acid molecules to be isolated admixed with other nucleic acid molecules, biomolecules, biological macrostructures or reagents. The first or second reagent contains a suitable concentration of a nucleic acid precipitating reagent (e.g., PEG) and salt to result in the facilitated absorption of the target species of nucleic acid molecule, but not of smaller-sized species of nucleic acid molecules or other biomolecules, biological macro structures or reagents present in the starting material.

Separation of the target species of nucleic acid molecule is accomplished by applying an external force (e.g., magnetic field, centrifigation, filtration) suitable to remove the solid phase carrier having the selectively precipitated nucleic acid bound thereto from the combination. In a preferred embodiment the solid phase carrier is a paramagnetic microparticle and separation is accomplished by applying a magnetic field of appropriate strength. In a further embodiment the solid phase carrier is a paramagnetic microparticle and separation is accomplished by applying a magnetic field of at least 1000 Gauss. This embodiment of the invention is useful for example to isolate a restriction enzyme digest fragment having a particular molecular size from smaller fragments present in the same digest; for isolating a single PCR product from a multiplex PCR reaction; for the selection of DNA fragments having homogenous sized distribution resulting from a shearing procedure (e.g., nebulizer, sonicator, hydroshear); for removing a nucleic acid template from a sequencing reaction or for selectively precipitating the extension products (e.g., Sanger Sequencing products) from a detemplated sequencing reaction prior to capillary electrophoresis. For example, the production of shattered DNA libraries for large scale sequencing experiments requires a size-selection step to minimize the deviation in size of the DNA inserts selected for cloning. The ability to produce a library comprising sheared DNA fragments, characterized by a narrow size distribution improves an investigator's ability to construct a map of the original pre-sheared DNA molecule. Using the method described herein an investigator can preselect a cut off size and formulate a binding buffer appropriate to precipitate and selectively adsorb a homogeneous population of DNA fragments. This embodiment can also be used to isolate extension products from a detemplated sequencing reaction mixture. The adsorbed nucleic acid molecules (e.g., Sanger sequencing products) can be thoroughly washed free of salts (e.g., reagent) and excess terminals whose presence will interfere with the electrophoretic injection of the sample to be sequenced.

In another embodiment, the method disclosed herein can be used to isolate two different species, for example, endogenous host cell nucleic acids and exogenous nucleic acid molecules, present in the starting material, by first isolating the relatively higher molecular weight host cell DNA, and subsequently isolating the relatively smaller-sized exogenous nucleic acid molecules. Thus, an additional embodiment of the instant invention further provides a means for the selective removal of endogenous host cell DNA by performing a first step designed to precipitate and promote the adsorption of host cell DNA chromosomal to the surface of a suitable solid phase carrier (e.g., a microparticle surface). According to this method, a first reagent described herein can be used if the starting material comprises a cell, or a second reagent described herein can be used if the starting material is a cell lysate. The removal of the solid phase carrier (to which the host cell DNA is bound) from the resulting mixture results in the removal of the relatively larger-sized host cell DNA.

As described above, high quality exogenous DNA can subsequently be isolated from an exogenous DNA enriched supernatant by selectively precipitating and adsorbing the relatively lower molecular weight exogenous DNA to the surfaces of additional solid phase carrier which is introduced resulting into the supernatant. A second reagent as described herein, with appropriate nucleic acid precipitating reagent concentrations, can be used to facilitate the isolation of the exogenous DNA.

An example of this additional embodiment can be performed by carrying out the following steps: combining functional group-coated paramagnetic microparticles and suitable concentrations of a precipitating reagent, for example, a polyalkylene glycol, and a salt to promote the facilitated adsorption of precipitated endogenous host cell nucleic acid (e.g., chromosomal DNA) and subsequently of exogenous nucleic acid molecules (e. g., bacterial or viral nucleic acids), each species being characterized by a particular molecular size, to the surfaces of the microparticles suspended therein; and the removal, such as by magnetic means, of the nucleic acid-coated microparticles from the resulting first combination. The removal of the microparticle having endogenous host cell nucleic acid adsorbed to its surfaces from the first combination results in the concomitant separation of host cell nucleic acid from both exogenous nucleic acid species and from other host cell biomolecules present in the sample. Exogenous nucleic acid present in the same sample can subsequently be isolated by producing a second combination by adding paramagnetic microparticles which have a functional group-coated surface and a sufficient quantity of a nucleic acid precipitating reagent to increase the concentration of the precipitating reagent to a level sufficient to result in the adsorption of exogenous nucleic acid to the microparticles suspended therein, thereby producing a third combination comprising exogenous nucleic acid bound to the microparticles; removing the paramagnetic microparticles from the third combination. Thus, exogenous nucleic acid bound to the microparticles is isolated from other host cell biomolecules present in the starting solution. Thus, the present invention also provides a method of selectively separating exogenous nucleic acids from relatively larger species of endogenous host cell nucleic acids present in the same sample.

The selective precipitation of endogenous host cell DNA (e.g., chromosomal or genomic DNA) can be mediated by concentrations of PEG as low as about 1% (w/v) and as high as about 4% (w/v) depending upon the size of the host cell DNA and the ionic strength of the solution. In a preferred embodiment, the concentration of PEG is preferably adjusted to about 3% (weight/volume). The subsequent selective precipitation of exogenous plasmid DNA is accomplished by adjusting the PEG concentration to a level which has been empirically determined to be optimal to promote the precipitation of a DNA species of a specified macromolecular size range. For example, exogenous DNA produced from the replication of a bacterial plasmid in a suitable strain of E. coli would be isolated by adjusting the PEG concentration of the second precipitation reaction to about 10% (weight/volume).

At high salt concentrations (e.g., synonymous with high ionic strengths) suitable paramagnetic microparticles will adsorb DNA fragments of all sizes. The term “high salt concentration” refers to salt concentrations greater than about 0.5 M.

At “low salt concentrations” (or low ionic strengths), which as used herein connotes concentrations less than about 0.2 M, essentially no DNA, of any size, will be precipitated even in the presence of a PEG concentration that is as high as 12% (weight/volume) (Lis, John T, Methods in Enzymology 65: 437-353 (1980). At intermediate salt concentrations (e.g., ranging from about 0.3M to about 0.45M) the characteristic macromolecular size of the DNA species precipitated by a particular PEG concentration is a function of the interaction between ionic strength and PEG concentration and reflects a relationship between macromolecular size and requisite threshold PEG concentration required to precipitate DNA molecules of a given size.

In general, smaller fragments of DNA will interact with the functional group-coated surfaces with a lower affinity than larger DNA fragments in the presence of relatively low concentrations of salt. To maximize yield and efficiency, sodium chloride concentration is preferably adjusted to about 0.55 M for the selective removal of host cell DNA from a lysed host cell suspension. Yields of bound DNA decrease if the salt concentration is adjusted to less than about 0.5 M or greater than about 5.0 M.

Purity (e.g. quality) of recombinant DNA isolated during the second precipitation reaction decreases if the sodium chloride concentration exceeds about 0.55 M.

Another embodiment of the instant invention provides a method by which recombinant nucleic acid molecules expressed in host cells can be selectively isolated from host cell lysates comprising a mixture of nucleic acid molecules and other host cell biomolecules. The following is a description of this embodiment with reference to nucleic acid molecules as exemplified by DNA. It is to be understood that the instant embodiment is also useful for separation of other nucleic acids in a similar manner. This embodiment of the invention comprises the steps of preparing a first combination comprising a lysed host cell solution prepared from cells expressing a recombinant nucleic acid; encapsulated carboxyl group coated paramagnetic microparticles, and low percentage PEG and low molarity salt.

According to the method disclosed herein, the PEG and salt are present at sufficient concentrations that high molecular weight host cell DNA is precipitated and reversibly binds (adsorbs) to the encapsulated carboxyl group-coated paramagnetic microparticles, thereby producing paramagnetic microparticles having host cell DNA bound thereto. The DNA-coated microparticles (and, thus, the microparticle adsorbed endogenous DNA) are removed from the first combination, thereby producing a recombinant DNA-enriched supernatant. A second combination is produced by adding carboxyl group-coated paramagnetic microparticles to the recombinant DNA-enriched supernatant and sufficient polyethyleme glycol to result in the selective precipitation and adsorption of the relatively smaller sized recombinant DNA to the surfaces of the micro-particles, thereby producing paramagnetic microparticles having recombinant DNA bound thereto; and removing the paramagnetic microparticles (and thus, the adsorbed recombinant DNA), whereby recombinant DNA is selectively isolated from host cell DNA.

Examples of recombinant DNA which can be introduced into a host cell include, but are not limited to, bacterial artificial chromosomes (BACs), yeast artificial chromosomes (YACs), PACs, Pls, cosmids and bacterial plasmids. The exogenous DNA may be directly introduced into a host cell, or an ancestor thereof, by means well known to one of ordinary skill in the art, such as transformation or transfection methods. Alternatively, plasmid DNA may be indirectly introduced into a host cell, or its ancestor by use of a phage into which exogenous DNA has been packaged. Suitable plasmid DNAs which can be packaged into a phage include a cosmid or P1 vector. Suitable host cells include bacterial cells, yeast cells, plant cells and mammalian cells. For example, suitable strains of E. coli bacteria include but are not limited to: DH5α, DH1, DH10B, DH12S, C600 or XL-1 Blue. As used herein the term “plasmid” refers to double stranded circular DNA species which originate from an exogenous source (e.g., are introduced into a host cell) and which are capable of self-replication independent of host chromosomal DNA. Thus, the term encompasses cloned DNA produced from the replication of any of the above mentioned vectors. Suitable vectors are well known in the art and include, for example high copy vectors, selected from, but not limited to the group consisting of pUC, pOT, pBluescript, pGEM, pTZ, pBR322, pSC101, pACYC, SuperCos and pWE15.

BACs are particularly difficult to separate and purify from cleared lysates due to their low concentrations in the lysates, which is attributable to their low copy number presence in the host cell. However, BAC DNA (e.g., up to 180 kb in size) is readily isolated by the method of the present invention. Cosmids are particularly difficult to isolate from expression host cells using commercially available chromatography-based methods because of their relatively large size (e.g., 35 to 40 kb). However, cosmids are readily separated by the methods of the present invention.

Thus, the method of the present invention is also useful to separate recombinant DNA resulting from the replication of an exogenous vector from a host cell lysate containing an admixture of host cell biomolecules, including host cell DNA and exogenous cloned DNA produced by the host cell. The simplicity and robust nature of the disclosed method makes it particularly useful for the preparation of DNA sequencing templates for automated nucleotide sequencing.

Another embodiment of the present invention relates to a method of isolating a nucleic acid molecule suitable for use as a template for nucleotide sequencing using either manual or high-throughput automated sequencing methods. This embodiment comprises: treating host cells with a first reagent as described herein; removing the paramagnetic microparticles having host cell DNA bound thereto from the suspension, thereby producing a plasmid-enriched supernatant; combining a second reagent as described herein; removing the microparticles having plasmid DNA bound thereto from the supernatant; washing the microparticles with a wash buffer to remove impurities adsorbed to the microparticles, thereby producing a purified template; and contacting the microparticle-bound purified template with an elution buffer, whereby the plasmid DNA template is released from the microparticles and is dissolved in the elution buffer, thereby isolating a purified plasmid DNA template suitable for nucleotide sequencing.

Kits and Automated Methods

The present invention further includes a kit comprising reagents for isolating nucleic acids from a variety of starting materials, e.g., cells or cell lysates. Kits described herein can be use for automated processing according to a system which has been optimized for high through-put DNA templates preparation.

The kit may comprise at least one composition described herein, e.g., a first and or second reagent. Additionally, the kit may comprise: at least one preformulated high ionic strength buffer suitable for use as a wash buffer, or reagents for preparing such buffer; a preformulated elution buffer or reagents for its preparation; a multisample vessel, e.g., a microtiter plate; and a magnetic multisample vessel holder, e.g., a microtiter plate holder, designed to optimize features of the magnetic field known to be crucial to the efficiency of automated processing. The design of the magnetic plate holder is instrumental in producing a magnetic field having the requisite, uniformity and field strength to maximize the efficiency achievable with automatic processing.

Field Strength of up to and over 1600 Gauss can be achieved with the use of N35 rare earth magnets configured with alternating North and South polar spaced 9 mm apart. Since the magnetically responsive microparticles are paramagnetic they will attract to either pole.

Placing magnets in one field orientation will extend a weaker field one meter from the plate. Opposite orientation magnetic poles create high magnetic fields localized to a 3 cm distance off of the plate(far enough to reach the samples). This results in fast separation times and is compliant with robotics.

A kit can be formulated for automated methods of isolating nucleic acids. For example, the kit can be formulated for use in a robotic device. The kit can contain a multisample transfer device, e.g., a multichannel pipette, used to transfer a reagent from a first vessel into a multisample holder, e.g. 96 well, 384, or 1536 formats. The kit can optionally contain a multisample transfer device preloaded with a reagent described herein, e.g. a first or second reagent. The kit can contain an additional multisample transfer device loaded with a different reagent described herein, e.g., a first or second reagent.

A kit can be formulated to perform any number of nucleic acid preparations, e.g., at least 1, 2, 3, 4, 5, 6, 7, 8, 9, 10, 20, 50, or 100. The kit may further include instructions related to methods of using the components contained therein, e.g., a first and or second reagent.

The following are examples of the practice of the invention. They are not to be construed as limiting the scope of the invention in any way.

### EXAMPLES

### Example 1

### Purification of Plasmid DNA

The following procedure was used to purify plasmid DNA from a host bacteria. Bacteria were grown in 200 μl 2×YT with 50 μg/ml chloramphenicol in a 384 well Greiner growth plate for 18 hours at 400 rpm shaking (with an airpore seal to prevent evaporation). A 384 well pipettor was used to aspirate 20 μl of cells directly following growth from the 384 well Greiner growth plate (an uncentrifuged growth plate was used to ensure that cells were not impacted).

20 μl of cells were dispensed to a well of a 384 well polystyrene plate. 85 μl of a first reagent was added to the well (the reagent included a lysis solution, a binding solution, and paramagnetic solid phase carriers). The final concentration of first reagent ingredients in the well were as follows: 3% polyethylene glycol 8000; 0.5 M NaCl; 0.2 N NaOH; 1% sodium dodecyl sulfate (SDS); and 0.0357% solids of COOH terminated paramagnetic particles. This solution simultaneously lyses the cells and binds the genomic (high molecular weight) E. coli DNA to magnetic particles including other insoluble material (e.g., proteins and endotoxin).

The 384 well polystyrene plate was placed on a magnetic plate for 5 minutes. Using a 384 pipettor, 73 μl of solution was aspirated at 3 μl/second, taking care not to disrupt the separated magnetic material. 73 μl was dispensed into a new 384 well polystyrene plate.

Next, 25 μl of a second reagent was added to the well (the reagent included a binding solution and paramagnetic solid phase carriers). The final concentration of second reagent ingredients in the well were as follows: 12.4% polyethylene glycol 8000(PEG); 0.37 M NaCl; and 0.04% solids of COOH terminated paramagnetic particles.

The 384 polystyrene well plate was placed on a magnetic plate for 5 minutes. Contaminants were removed using a 70% EtOH wash solution. Plasmid DNA bound to the magnetic particles was removed by the addition of 15 μl of aqueous solution. 3 μl of DNA-containing solution was removed for sequencing.

### Example 2

### Sequencing of Purified Plasmid DNA

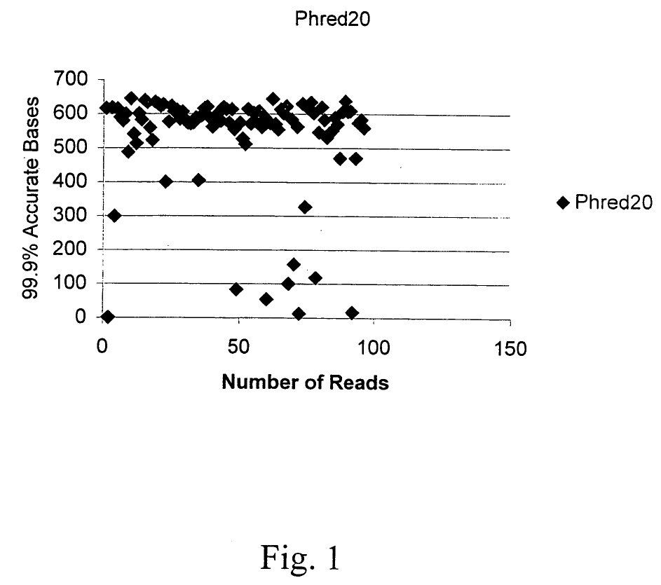

FIG. 1 is a graph depicting the sequencing results of the plasmid purified in Example 1, as detected on a PE 3700 Capillary DNA sequencing with 1/16^{th} dilution of the manufacturer's recommended Big Dye Sequencing reagent. The results attained with 1/16^{th} dilution suggests the DNA is of high quality and suitable for sequencing.

### Example 3

### Purification of Plasmid DNA Using a 96 Well Format

The following procedure can be used to purify plasmid DNA from host bacteria using a 96 well format. 50 μl of bacterial cell culture is dispensed into a well of a 96 well plate. 230 μl of a first reagent is added to the well (the reagent includes a lysis solution, a binding solution, and paramagnetic solid phase carriers). The mixture is optionally pipetted up and down three times. The final concentration of first reagent ingredients in the well are as follows: 3% polyethylene glycol 8000; 0.5 M NaCl; 0.2 N NaOH; 1% sodium dodecyl sulfate (SDS); and 0.0357% solids of COOH terminated paramagnetic particles.

The 96 well plate is placed on a magnetic plate for 8 minutes. Using a 96 pipettor, 200 μl of solution is aspirated at 2 μl/second, taking care not to disrupt the separated magnetic material. 200 μl is dispensed into a new 96 well plate.

Next, 80 μl of a second reagent is added to the well (the reagent includes a binding solution and paramagnetic solid phase carriers). The mixture is optionally pipetted up and down three times. The final concentration of second reagent ingredients in the well is as follows: 12.4% polyethylene glycol 8000(PEG); 0.37 M NaCl; and 0.04% solids of COOH terminated paramagnetic particles.

The 96 polystyrene well plate is placed on a magnetic plate for 8 minutes. Contaminants are removed by performing four washes using a 70% EtOH wash solution. Plasmid DNA bound to the magnetic particles is removed by the addition of an aqueous solution.

What is claimed is:

1. A method of isolating a first species of nucleic acid molecule from a cell, the method comprising the steps of:
(a) providing a cell;
(b) preparing a first combination by simultaneously adding to the cell one or more reagent components collectively referred to as a first reagent, wherein the first reagent is formulated to cause lysis of the cell, wherein the first reagent comprises a nucleic acid precipitating reagent and a solid phase carrier having a surface that binds nucleic acid molecules, wherein the nucleic acid precipitating reagent is present in sufficient concentration to precipitate the first species of nucleic acid molecule;
(c) maintaining the first combination under conditions that permit the adsorption of the precipitated nucleic acid molecule to the solid phase carrier, thereby producing a solid phase carrier having bound thereto the first species of nucleic acid molecule; and
(d) removing the carrier having bound thereto the first species of nucleic acid molecule from the first combination, thereby isolating the first species of nucleic acid molecule from the mixture and producing a second mixture.

2. The method of claim 1, wherein the nucleic acid precipitating reagent of the first reagent is a polyalkylene glycol.

3. The method of claim 2, wherein the polyalkylene glycol is polyethylene glycol (peg).

4. The method of claim 1, wherein the first reagent is added to the cell by a multisample transfer device.

5. The method of claim 1, wherein the solid phase carrier of the first reagent is a magnetically responsive solid phase carrier.

6. The method of claim 5, wherein the solid phase carrier comprises a functional group coated surface.

7. The method of claim 6, wherein the solid phase carrier is an amine-coated paramagnetic microparticle.

8. The method of claim 6, wherein the solid phase carrier is a carboxyl-coated paramagnetic microparticle.

9. The method of claim 6, wherein the solid phase carrier is an encapsulated carboxyl group-coated paramagnetic microparticle.

10. The method of claim 6, wherein the pH of the first reagent is formulated to adjust the binding affinity of the functional group to the first species of nucleic acid molecule.

11. The method of claim 1, wherein the solid phase carrier of the first reagent is removed by applying a magnetic field, applying vacuum filtration, or by centrifugation.

12. The method of claim 3, wherein the first reagent further comprises a salt selected from the group consisting of sodium chloride, magnesium chloride, calcium chloride, potassium chloride, lithium chloride, barium chloride and cesium chloride.

13. The method of claim 1, wherein the first reagent consists of one reagent component.

14. The method of claim 1, wherein the solid phase carrier reversibly binds nucleic acid molecules.

15. The method of claim 1, further comprising isolating a second species of nucleic acid molecules from the second mixture, the method further comprising the steps of:
(e) preparing a second combination by simultaneously adding to the second mixture one or more reagent components collectively referred to as a second reagent, wherein the second reagent comprises a nucleic acid precipitating reagent and a solid phase carrier having a surface that binds nucleic acid molecules, wherein the precipitating reagent is present in sufficient concentrations to precipitate the second species of nucleic acid molecule;
(f) maintaining the second combination under conditions appropriate for the adsorption of the second target species of nucleic acid molecule to the surface of the solid phase carrier, thereby producing a solid phase carrier having the second species of nucleic acid molecule bound thereto; and
(g) removing the solid phase carrier having the second species of nucleic acid molecule adsorbed thereto from the second combination.

16. The method of claim 15, further comprising the step of:
(h) eluting the second species of nucleic acid molecules from the solid phase carrier, thereby selectively isolating an second species of nucleic acid molecules.

17. The method of claim 15, wherein the second species is of a smaller molecular size than the first species removed in step (d).

18. The method of claim 15, wherein the nucleic acid precipitating reagent of the second reagent is a polyalkylene glycol.

19. The method of claim 18, wherein the polyalkylene glycol is PEG.

20. The method of claim 15, wherein the second reagent is added to the second mixture by a multisample transfer device.

21. The method of claim 15, wherein the solid phase carrier of the second reagent is a magnetically responsive solid phase carrier.

22. The method of claim 21, wherein the solid phase carrier comprises a functional group coated surface.

23. The method of claim 22, wherein the solid phase carrier is an amine-coated paramagnetic microparticle.

24. The method of claim 22, wherein the solid phase carrier is a carboxyl-coated paramagnetic microparticle.

25. The method of claim 22, wherein the solid phase carrier is an encapsulated carboxyl group-coated paramagnetic microparticle.

26. The method of claim 15, wherein the solid phase carrier of the second reagent is removed by applying a magnetic field, applying vacuum filtration, or by centrifugation.

27. The method of claim 19, wherein the second reagent further comprises a salt selected from the group consisting of sodium chloride, magnesium chloride, calcium chloride, potassium chloride, lithium chloride, barium chloride and cesium chloride.

28. The method of claim 15, wherein the second reagent consists of one reagent component.

29. The method of claim 15, wherein the solid phase carrier reversibly binds nucleic acid molecules.

30. The method of claim 20, wherein the first reagent comprises polyethylene glycol and salt in concentrations that result in the binding of the first species of nucleic acid molecule to the solid phase carrier in step (c), but does not result in the binding of the second species of nucleic acid molecule to the solid phase carrier in step (c).

31. The method of claim 20, wherein the polyethylene glycol has an average molecular weight of about 8,000, and the polyethylene glycol concentration of the first combination is between about 1% and 4% and the polyethylene glycol concentration of the second combination is at least 7%.

32. A method of isolating a nucleic acid molecule from a cell, the method comprising adding to the cell one or more reagent components collectively referred to as a first reagent, wherein the first reagent causes the lysis of the cell and comprises a nucleic acid precipitating reagent and a solid phase carrier having a surface that reversibly binds a nucleic acid molecule of the cell.

33. The method of claim 32, further comprising removing the solid phase carrier with a first species of nucleic acid attached thereto, to generate a first mixture.

34. The method of claim 33, further comprising adding to the first mixture one or more reagent components collectively referred to as a second reagent, wherein the second reagent comprises a nucleic acid precipitating reagent and a solid phase carrier having a surface that reversibly binds a nucleic acid molecule of the cell.

35. A composition for isolating nucleic acids, wherein the composition comprises a nucleic acid precipitating reagent and a solid phase carrier having a surface that binds nucleic acid molecules, wherein the composition is formulated to cause the lysis of a cell, wherein the composition lacks one or more of nucleic acids, cells, or cellular lysate.

36. The composition of claim 35, wherein the composition comprises PEG and salt.

37. The-composition of claim 35, wherein the PEG and salt are present in sufficient concentration to selectively precipitate nucleic acid molecules greater 10 kb when the composition is added to a cell.

38. The composition of claim 36, wherein the concentration of PEG is formulated to be between about 1-4% when the composition is added to a cell.

39. The composition of claim 36, wherein the concentration of salt is formulated to be at least 0.5M when the composition is added to a cell.

40. The composition of claim 34, wherein the pH of the composition is formulated to adjust the binding affinity of the surface of the solid phase carrier to nucleic acid molecules.

41. A composition for isolating nucleic acids, wherein the composition comprises a nucleic acid precipitating reagent and a solid phase carrier having a surface that reversibly binds nucleic acid molecules, wherein the composition lacks one or more of nucleic acids, cells, or cellular lysate.

42. The composition of claim 41, wherein the composition comprises PEG and salt.

43. The composition of claim 42, wherein the PEG and salt are present in sufficient concentration to selectively precipitate nucleic acid molecules greater 2.4 kb when the composition is added to a cell.

44. The composition of claim 43, wherein the concentration of PEG is formulated to be at least 7% when the composition is added to a cell.

45. The composition of claim 43, wherein the concentration of salt is formulated to be between less than 055M when the composition is added to a cell.

46. A kit for isolating nucleic acids, comprising:
a first composition, wherein the first composition comprises a nucleic acid precipitating reagent and a solid phase carrier having a surface that binds nucleic acid molecules, wherein the first composition is formulated to cause the lysis of a cell, wherein the first composition lacks one or more of nucleic acids, cells, or cellular lysate; and
a second composition, wherein the second composition comprises a nucleic acid precipitating reagent and a solid phase carrier having a surface that reversibly binds nucleic acid molecules, wherein the second composition lacks one or more of nucleic acids, cells, or cellular lysate.

47. The kit of claim 46, further comprising a third composition and a fourth composition, wherein the third composition dissolves impurities but not nucleic acids bound to a solid phase carrier, and wherein the fourth composition is a low ionic strength buffer.

48. The kit of claim 46, further comprising a magnetic plate holder appropriate for applying a magnetic field of at least about 1000 Gauss to the wells of a microtiter plate, wherein the magnet comprises at least one N35 magnet.
